(19) United States
(12) Patent Application Publication
(10) Pub. No.: US 2025/0261984 A1
(43) Pub. Date: Aug. 21, 2025

(54) ABLATION CATHETERS AND SYSTEMS

(71) Applicant: Aqua Therapeutics, Inc., Santa Ana, CA (US)

(72) Inventors: George W. Bourne, Boston, MA (US); Virender K. Sharma, Paradise Valley, AZ (US); Nathalie Perry, Cambridge, MA (US); Amos Cruz, Wrentham, MA (US); Minh Duong, Worcester, MA (US)

(21) Appl. No.: 19/060,543

(22) Filed: Feb. 21, 2025

Related U.S. Application Data
(60) Provisional application No. 63/556,349, filed on Feb. 21, 2024.

(51) Int. Cl. A61B 18/04; A61B 18/00

(52) U.S. Cl. CPC A61B 18/04; A61B 2018/00559; A61B 2018/00577; A61B 2018/0072; A61B 2018/00744; A61B 2018/0094; A61B 2018/00946; A61B 2018/048; A61B 2218/007

(57) ABSTRACT

An ablation catheter and an ablation system for ablating a uterine cavity of a patient. The catheter has ablation ports configured between two positioning elements and at least one port at the center of an atraumatic distal tip. An internal heating chamber is disposed within a lumen of the catheter to heat a fluid and generate vapor. The internal heating chamber comprises an electrode or an array of electrodes that are positioned circumferentially around a central core.

### CROSS-REFERENCE TO RELATED APPLICATIONS

This application claims priority to U.S. Provisional Application No. 63/556,349 filed on Feb. 21, 2024, the entire contents of which are incorporated herein by reference.

### FIELD

The present specification relates to systems and methods configured to generate and deliver vapor for ablation therapy, as well as methods of use of the same.

### BACKGROUND

Ablation, as it pertains to the present specification, relates to the removal or destruction of a body tissue, via the introduction of a destructive agent, such as radiofrequency energy, laser energy, ultrasonic energy, cyroagents, or steam. Ablation is commonly used to eliminate diseased or unwanted tissues, such as, but not limited to cysts, polyps, tumors, hemorrhoids, and other similar lesions. Ablation techniques may be used in combination with chemotherapy, radiation, surgery, and Bacillus Calmette-Guérin (BCG) vaccine therapy, among others.

Steam-based ablation systems, such as the ones disclosed in U.S. Pat. Nos. 9,615,875, 9,433,457, 9,376,497, 9,561,068, 9,561,067, and 9,561,066, disclose ablation systems that controllably deliver steam through one or more lumens toward a tissue target. One problem that all such steam-based ablation systems have is the potential overheating or burning of healthy tissue. Steam passing through a channel within a body cavity heats up surfaces of the channel and may cause exterior surfaces of the medical tool, other than the operational tool end itself, to become excessively hot. As a result, physicians may unintentionally burn healthy tissue when external portions of the device, other than the distal operational end of the tool, accidentally contacts healthy tissue. U.S. Pat. Nos. 9,561,068, 9,561,067, and 9,561,066 are hereby incorporated herein by reference.

It is desirable to have steam-based ablation devices that integrate into the device itself safety mechanisms which prevent unwanted ablation during use. It is further desirable to have a catheter handle that allows the user to ergonomically hold the device during a vapor ablation treatment. It also desirable to have steam-based ablation devices, and methods of use and treatment related to the same, which deliver strong, steady, and reliable flows of vapor.

### SUMMARY

The present specification discloses embodiments of an ablation catheter, which may be used for ablating endometrial tissue. The catheter may include an outer sheath with at least one opening, an inner shaft with at least one lumen configured to receive a volume of fluid, at least one positioning element, at least one port, a tip positioned the inner shaft, at least one heating component within the lumen, and a handle coupled to the inner shaft. The specification also discloses embodiments of an ablation system, which may include a disclosed embodiment of a catheter in addition to a fluid reservoir, a pump, and a controller. Also disclosed are embodiments of a kit, which includes multiple components of an embodiment of an ablation system.

The aforementioned and other embodiments of the present invention shall be described in greater depth in the drawings and detailed description provided below.

### BRIEF DESCRIPTION OF THE DRAWINGS

These and other features and advantages of the present invention will be further appreciated, as they become better understood by reference to the detailed description when considered in connection with the accompanying drawings.

FIG. 1 is a photograph of an embodiment of an ablation system for ablating a uterine cavity of a patient.

FIG. 2 illustrates, with annotations, an embodiment of a generator system used by the ablation system shown in FIG. 1.

FIG. 3 illustrates an embodiment of a catheter system used by the ablation system of FIG. 1.

FIG. 4 illustrates a top view of an embodiment of a catheter handle as part of an embodiment of a catheter system.

FIG. 5 illustrates a view of internal components of an embodiment of a catheter handle.

FIG. 6 illustrates a close-up view of a distal positioning element slider attachment to proximal shaft of the catheter handle of FIG. 5.

FIG. 7 illustrates an enlarged view of an outer sheath slider attachment to the outer sheath of the catheter handle of FIG. 5.

FIG. 8 illustrates a view of the first slider attached to a proximal end of an attachment of the catheter handle of FIG. 5.

FIG. 9 illustrates a view of the second slider attached to proximal end of an attachment of the catheter handle of FIG. 5.

FIG. 10 illustrates a cross-section view along a longitudinal axis of an embodiment of a catheter.

FIG. 11 illustrates a perspective view of an embodiment of a cervical stabilizer.

FIG. 12 illustrates a top view of the cervical stabilizer of FIG. 11.

FIG. 13 illustrates a side view of the cervical stabilizer of FIG. 11.

FIG. 14 illustrates a front view of an embodiment of a cervical stabilizer with internal ribbing.

FIG. 15 illustrates a view of an embodiment of a cervical stabilizer as part of an embodiment of a catheter system.

FIG. 16 illustrates a cross-section view of an embodiment of a catheter lumen.

FIG. 17 illustrates an enhanced view of the area identified as “A-A” on FIG. 16.

FIG. 18 illustrates an enhanced view of the area identified as “B-B” on FIG. 16.

FIG. 19 illustrates an enhanced view of the area identified as “C-C” on FIG. 16.

FIG. 20 illustrates side perspective view of an embodiment of a distal tip of a catheter lumen.

FIG. 21 illustrates a transverse cross-section view of a first array of electrodes and a second array of electrodes as may be employed with disclosed embodiments of a catheter system.

FIG. 22 illustrates a side view of the arrays of electrodes in FIG. 21 annotated with dimensions in accordance with a disclosed embodiment.

FIG. 23 illustrates a side view of an embodiment of a catheter shaft.

FIG. 24 illustrates a cross-section view of the catheter shaft of FIG. 23.

FIG. 25 illustrates an embodiment of a set of insulation layers used in an ablation catheter shaft.

FIG. 25A is a graph chart showing insulation temperature as compared to the number of wraps of insulation.

FIG. 26 illustrates an embodiment of a cervical stabilizer over an embodiment of a uterine aspiration catheter assembly.

FIG. 27 illustrates an enhanced view of the area identified as “D-D” on FIG. 26.

FIG. 28 illustrates an exploded side view of an embodiment of a cervical stabilizer over an embodiment of a uterine aspiration catheter assembly annotated with dimensions in accordance with an embodiment a disclosed embodiment.

FIG. 29 illustrates multiple components forming the ablation system of the present specification, packaged together in a kit.

FIG. 30 illustrates another view of the packaging elements of the kit, in accordance with some embodiments of the present specification.

FIG. 31 is a flow chart illustrating a method for preparing and using the ablation catheter device or system described in accordance with the various embodiments of the present specification.

FIG. 32 shows an embodiment of a catheter positioned within a uterus.

FIG. 33 shows the catheter of FIG. 33 with vapor being released in accordance with an embodiment of a uterine ablation procedure.

FIG. 34 is a graph showing temperature in the uterine cavity, the cervical canal and the external fundus, as measured at different points in time during a uterine ablation procedure using disclosed embodiments.

### DETAILED DESCRIPTION

As used in this disclosure, the terms “treat,” “treatment,” and variations thereof refer to any reduction in the extent, frequency, or severity of one or more symptoms or signs associated with a condition. The term “duration” and variations thereof refer to the time course of a prescribed treatment, from initiation to conclusion, whether the treatment is concluded because the condition is resolved or the treatment is suspended for any reason. Over the duration of treatment, a plurality of treatment periods may be prescribed during which one or more prescribed stimuli are administered to the subject. The term “period” refers to the time over which a “dose” of stimulation is administered to a subject as part of the prescribed treatment plan. The term “controller” refers to an integrated hardware and software system defined by a plurality of processing elements, such as integrated circuits, application specific integrated circuits, and/or field programmable gate arrays, in data communication with memory elements, such as random access memory or read only memory where one or more processing elements are configured to execute programmatic instructions stored in one or more memory elements. The term “vapor generation system” refers to any or all of the heater or induction-based approaches to generating steam from water described in this application.

As used herein, each of the words “comprise” “include” and “have”, and forms thereof, are not necessarily limited to members in a list with which the words may be associated. The terms “comprises” and variations thereof do not have a limiting meaning where these terms appear in the description and claims. The term “and/or” means one or all of the listed elements or a combination of any two or more of the listed elements. Unless otherwise specified, “a,” “an,” “the,” “one or more,” and “at least one” are used interchangeably and mean one or more than one.

Embodiments of the present specification are useful in the treatment of uterine tissue, but it is contemplated within the disclosure that embodiments herein may be used for treatment of genitourinary structures, where the term “genitourinary” includes all genital and urinary structures, including, but not limited to, the prostate, uterus, and urinary bladder, and any conditions associated therewith, including, but not limited to, benign prostatic hyperplasia (BPH), prostate cancer, uterine fibroids, abnormal uterine bleeding (AUB), overactive bladder (OAB), strictures, and tumors.

Any and all of the needles and needle configurations disclosed in the specification with regards to a particular embodiment, such as including but not limited to, single needles, double needles, multiple needles and insulated needles, are not exclusive to that embodiment and may be used with any other of the embodiments disclosed in the specification in any of the organ systems for any condition related to the organ system such as and not limited to ablation of prostate, uterus, and bladder.

For purposes of the present specification, ‘completely ablating’ is defined as ablating more than 55% of a surface area or a volume around an anatomical structure.

All of the methods and systems for treating the prostate, uterus, and bladder may include optics or visualization as described in the specification to assist with direct visualization during ablation procedures.

For any method disclosed herein that includes discrete steps, the steps are presumably performed in order each is introduced or described, but it is contemplated within the scope of the disclosure that the steps may be conducted in any feasible order unless specified otherwise. And, as appropriate, any combination of two or more steps may be conducted simultaneously.

Also herein, the recitations of numerical ranges by endpoints include all numbers subsumed within that range (e.g., 1 to 5 includes 1, 1.5, 2, 2.75, 3, 3.80, 4, 5, etc.). Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present specification. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the specification are approximations, the numerical values set forth in the specific examples are reported as precisely as possible. All numerical values, however, inherently contain a range in necessarily resulting from the standard deviation found in their respective testing measurements.

The devices and methods of the present specification can be used to cause controlled focal or circumferential ablation of targeted tissue to varying depth in a manner in which complete healing with re-epithelialization can occur. Additionally, the vapor could be used to treat/ablate benign and malignant tissue growths resulting in destruction, liquefaction and absorption of the ablated tissue. The dose and manner of treatment can be adjusted based on the type of tissue and the depth of ablation needed. The ablation devices can be used for the prostate and endometrial ablation and for the treatment of any mucosal, submucosal or circumferential lesion, such as inflammatory lesions, tumors, polyps and vascular lesions. The ablation devices can also be used for the urinary bladder ablation, and for treating an over-active bladder (OAB). The ablation device can also be used for the treatment of focal or circumferential mucosal or submucosal lesions of the genitourinary tract. The ablation device can be placed endoscopically, radiologically, surgically or under direct visualization. In various embodiments, wireless endoscopes or single fiber endoscopes can be incorporated as a part of the device. In another embodiment, magnetic or stereotactic navigation can be used to navigate the catheter to the desired location. Radio-opaque or sonolucent material can be incorporated into the body of the catheter for radiological localization. Ferromagnetic materials can be incorporated into the catheter to help with magnetic navigation.

Ablative agents such as steam, heated gas or cryogens, such as, but not limited to, liquid nitrogen are inexpensive and readily available and are directed via the infusion port onto the tissue, held at a fixed and consistent distance, targeted for ablation. This allows for uniform distribution of the ablative agent on the targeted tissue. The flow of the ablative agent is controlled by a microprocessor according to a predetermined method based on the characteristic of the tissue to be ablated, required depth of ablation, and distance of the port from the tissue. The microprocessor may use temperature, pressure or other sensing data to control the flow of the ablative agent. In addition, one or more suction ports are provided to suction the ablation agent from the vicinity of the targeted tissue. The targeted segment can be treated by a continuous infusion of the ablative agent or via cycles of infusion and removal of the ablative agent as determined and controlled by the microprocessor.

It should be appreciated that the devices and embodiments described herein are implemented in concert with a controller that comprises a microprocessor executing control instructions. The controller can be in the form of any computing device, including desktop, laptop, and mobile device, and can communicate control signals to the ablation devices in wired or wireless form.

The present invention is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

It should be noted herein that any feature or component described in association with a specific embodiment may be used and implemented with any other embodiment unless clearly indicated otherwise.

One embodiment disclosed is an ablation catheter, which may be used for ablating endometrial tissue of a patient. The catheter may include an outer sheath having a first diameter and a length extending between a proximal end and a distal end, the outer sheath configured with at least one opening. The catheter may further include an inner shaft having at least one lumen, wherein the lumen is configured to receive a volume of fluid. At least one positioning element may be positioned proximate a proximal or distal end of the inner shaft, wherein the at least one positioning element is configured to expand from a first compressed delivery configuration to a second expanded deployed configuration. At least one first port positioned may be on the inner shaft. An atraumatic tip may be positioned on a distal end of the inner shaft, wherein the atraumatic tip comprises at least one second port in fluid communication with the inner shaft. At least one heating component may be positioned within the lumen, and the at least one heating component may include at least one electrode positioned circumferentially around a central core, and the at least one heating component may be configured to convert the volume of fluid received by the lumen to a vapor that exits from the at least one first port and the at least one second port. A handle may also be coupled to a proximal end of the inner shaft.

In further embodiments of an ablation catheter, the at least one electrode may include at least one array of electrodes. The array of electrodes may include at least two electrodes having tapered proximal ends, and the at least two electrodes may be configured to be positioned circumferentially around the central core such that the two tapered proximal ends are offset from one another. A wall of the inner shaft may include at least one insulation layer. The core may tapered at a proximal end of the core. The at least one positioning element may include a wire mesh structure. The handle may include at least one sliding button to control movement of the sheath to control deployment of the at least one positioning element.

Embodiments of an ablation catheter may further include a stabilizer positioned coaxially around an outer circumference of the sheath on a proximal side of the first positioning element. The stabilizer may configured to stabilize and maneuver the sheath during insertion inside the uterine cavity. The stabilizer may include a first proximal section, a second section attached to and perpendicular the first section, a third section attached to a distal end of, and at an angled orientation relative to, the second section, where the third section extends in a distal direction relative to the second section, and finally a fourth section extending in a distal direction relative to the second section, where the fourth section is attached to a side of a distal end of the third section, and the fourth section is oriented parallel to the second section, and wherein the fourth section is configured to be hollow to accommodate passage of the sheath.

Embodiments of an ablation system are also disclosed herein. The ablation system may be for ablating endometrial tissue of a patient. The ablation system may include an embodiment of a catheter disclosed herein. The ablation system may further include a fluid reservoir configured to contain the volume of fluid and in fluid communication with the at least one lumen. The ablation system may include a pump configured to pump fluid from the fluid reservoir into the at least one lumen. The ablation system may also include a controller having at least one processor, wherein the controller is configured to deliver an electrical current to the at least one electrode and to cause the volume of fluid to pass into the lumen from the fluid reservoir when activated.

In further embodiments of an ablation system, the system may include a power source positioned in the controller. The pump may include a syringe pump. The at least one electrode may include at least one array of electrodes. The core may be tapered at a proximal end of the core. The controller may include a foot pedal or switch configured to allow a user to control a flow of fluid to the lumen. The at least one positioning element may include a wire mesh structure. The handle may include at least one sliding button to control movement of the sheath to control deployment of the at least one positioning element. Embodiments of an ablation system may further include a stabilizer positioned coaxially around an outer circumference of the sheath on a proximal side of the first positioning element. The stabilizer may configured to stabilize and maneuver the sheath during insertion inside the uterine cavity. The stabilizer may include a first proximal section, a second section attached to and perpendicular the first section, a third section attached to a distal end of, and at an angled orientation relative to, the second section, where the third section extends in a distal direction relative to the second section, and finally a fourth section extending in a distal direction relative to the second section, where the fourth section is attached to a side of a distal end of the third section, and the fourth section is oriented parallel to the second section, and wherein the fourth section is configured to be hollow to accommodate passage of the sheath.

Embodiments of a kit are also disclosed herein, where the kit includes multiple components of an ablation system. The kit may include an embodiment of an ablation catheter. The kit may further include at least one pump configured to pump fluid from a fluid reservoir into the at least one lumen. The kit may also include an aspiration catheter. The kit may include a substrate having a plurality of spaces configured to receive the ablation catheter, at least one pump, and aspiration catheter, where at least one first cover portion of the substrate is configured to cover and protect at least one component of the ablation catheter. The kit may include a base configured to receive the substrate. The kit may include a lid configured to be positioned over the substrate and secured to the base.

In further embodiments of a kit, the at least one first cover portion may be partially cut out from a remainder of the substrate and formed by folding the at least one first cover portion over the at least one component of the ablation catheter. The kid may include at least one second cover portion and at least one third cover portion, where the at least one first, second, and third cover portions are configured to engage with each other to form an enclosure to cover the at least one component of the ablation catheter. The lid may include a plurality of recessed cavities that are recessed according to shapes of the ablation catheter, at least one pump, and aspiration catheter. At least one space of the plurality of spaces of the substrate may be configured to receive the ablation catheter comprising the at least one positioning element configured to expand from a first compressed delivery configuration to a second expanded deployed configuration, wherein the at least one positioning element is in the second expanded deployed configuration when the ablation catheter is positioned within the at least one space, and at least one first cover portion may be configured to cover and protect the at least one positioning element in the second expanded deployed configuration.

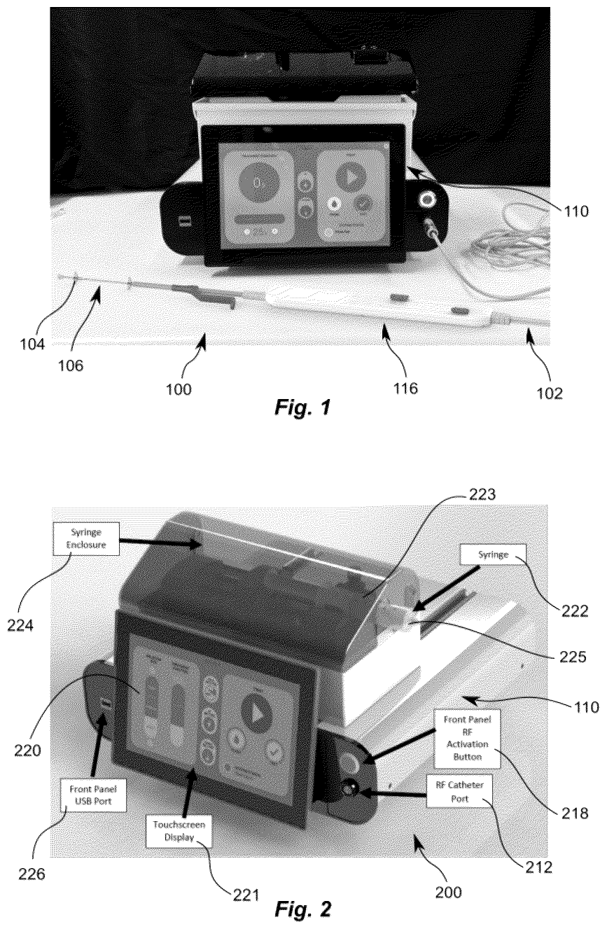

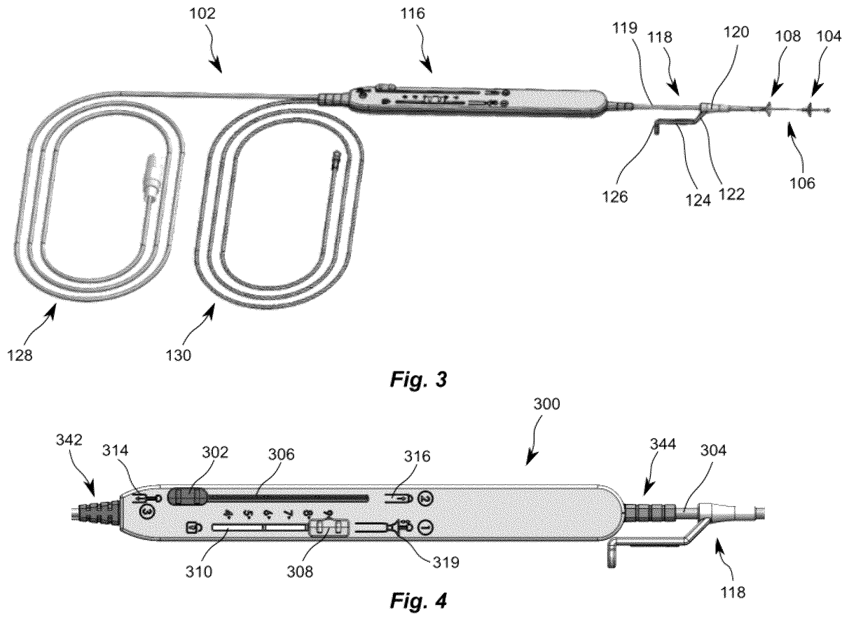

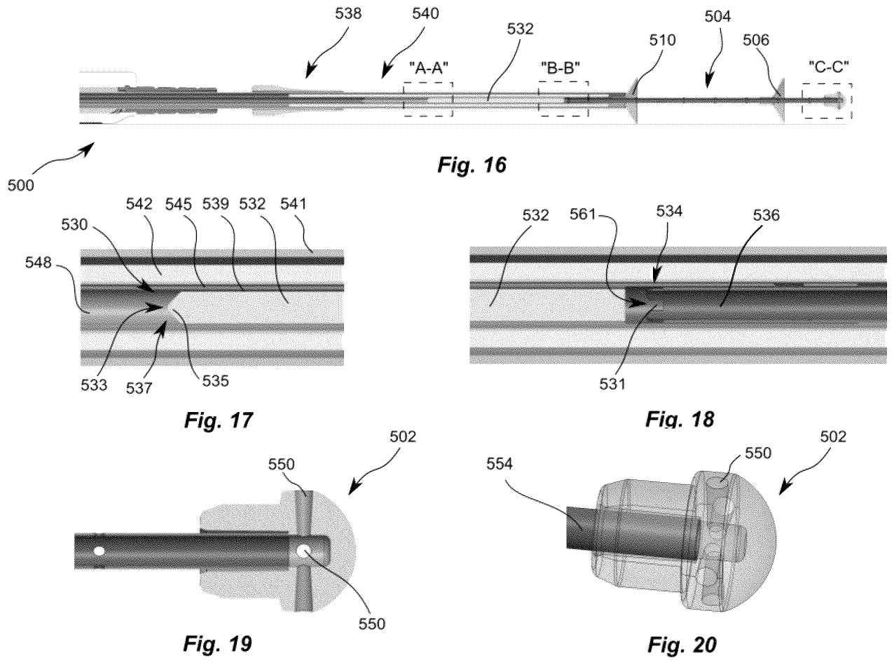

With reference now to the drawings, FIG. 1 illustrates an ablation system 100 for ablating a uterine cavity of a patient, in accordance with some embodiments of the present specification. FIG. 2 illustrates an embodiment of a generator system 200 used in ablation system 100, in accordance with the present specification. FIG. 3 illustrates an embodiment of a catheter 102 used in ablation system 100, in accordance with the present specification. Referring simultaneously to FIGS. 1-3, ablation system 100 comprises a catheter 102 having at least one attachment or positioning element 104 and an internal heating chamber, further described with reference to FIGS. 16-18, 21, and 22, disposed within a lumen of catheter 102 and configured to heat a fluid provided to catheter 102 to change said fluid to a vapor for ablation therapy. The internal heating chamber comprises an electrode or an array of electrodes that, in some embodiments, are positioned circumferentially, or wrapped about, a central core and are separated from thermally conductive elements by a segment of catheter 102 which is electrically non-conductive. In some embodiments, each electrode and the core are configured with tapered proximal ends to help prevent electrode shorting. Additionally, the tapered configuration of the proximal end of the electrode(s) and core ensures that flow of ablation fluid over the electrode(s) is less turbulent, relative to that offered by non-tapered configurations. As an ablation fluid approaches a tapered edge indicating the start of the electrode, the fluid's flow is rendered laminar thus resulting in less turbulence and more consistent contact across electrode surface. In some embodiments, catheter 102 is made of or covered with an insulated material to prevent the escape of ablative energy from the catheter body.

Catheter 102 comprises one or more delivery ports 106 for the delivery of ablative agent, such as steam. In some embodiments, catheter 102 includes a second positioning element 108 proximal to the first positioning element 104. In some embodiments, the one or more delivery ports 106 are positioned along the catheter between the first positioning element 104 and the second positioning element 108. In some embodiments, one or more additional delivery ports 106 are positioned along the catheter distal to the first positioning element 104. In various embodiments, the first distal attachment or positioning element 104 and second positioning element 108 may be any one of a disc, hood, cap, or inflatable balloon. Embodiments of ablation catheter 102 are packaged and shipped with the two positioning elements 104 and 108 being unsheathed such that they are in an expanded configuration, which is the configuration assumed by the positioning elements 104 and 108 during deployment within the uterine cavity. The positioning elements are packaged and shipped unsheathed since, in compressed form, there is a possibility that, when the positioning elements are subsequently unsheathed, they might not expand as a result of a sterilization process which, if done on the positioning elements in their compressed configurations, could cause them to remain compressed. When expanded, the second, or proximal, positioning element 108 provides tactile feedback and both the second or proximal positioning element 108 and first, or distal, positioning element 104 position the catheter in a uterus such that the one or more delivery ports provide steam diffusion through uterine cavity. In some embodiments, the first and second positioning elements have a diameter of approximately 1.5 cm in their expanded/deployed states. Both positioning elements 104 and 108 provide scaffolding to ensure there is spacing from delivery port(s) to uterine cavity surface tissue (to avoid blocking of the delivery ports). Additionally, in the deployed (expanded) position, the positioning elements 104 and 108 enable centering of the delivery ports within the volume of the uterine cavity. In embodiments, the delivery ports are provided between the two positioning elements, on a distal side of the distal positioning element, and in the distal tip. In some embodiments, the first positioning element 104 and second positioning element 108 have a wire mesh structure with or without a covering membrane. In some embodiments, the first distal attachment or positioning element 104 and second positioning element 108 include pores for the escape of air or ablative agent. A handle 116 provides slider mechanisms to control the movement of an outer sheath, which when pulled back enables deployment (expansion) of the positioning elements and, optionally, to control the positioning of distal positioning element 104.

In some alternate embodiments, a needle catheter is provided (not shown). The needle catheter uses a straight needle for treatment of fibroids within the uterine cavity. The needle catheter enables mechanical pre-dilating of a tunnel or provides a jetting effect of vapor which is delivered at a high pressure to cut tissue.

FIG. 2 illustrates another view of a generator 200 in accordance with some embodiments of the present specification. FIG. 3 illustrates a view of a catheter 102 with handle 116. Referring simultaneously to FIGS. 1-3, a fluid, such as saline, is stored in a reservoir, such as a saline pump attached to generator 200, connected through syringe 222 to a fluid line 130 of catheter 102. Additionally, an RF cable 128 connects RF catheter port 212 to controller 110. Delivery of the ablative agent is controlled by controller 110 of the generator 200 and treatment is controlled by a treating physician via the controller 110. Prior to a treatment, the entire ablation system is primed, and vapor is generated and passed through the infusion ports momentarily. The test shot is performed after priming, and prior to starting the treatment, to ensure that the ablation system is generating steam. The generator 200 includes a controller 110 comprising at least one processor in data communication with the saline pump. The generator system 200 also includes an RF catheter connection port 212 and the controller 110 is configured to control electrical current delivery to the electrodes of the catheter via the RF catheter connection port 212 and fluid delivery to the catheter via the saline pump and syringe 222. In some embodiments, at least one optional sensor monitors changes in an ablation area to guided flow of ablative agent. In some embodiments, the optional sensor comprises at least one of a temperature sensor or pressure sensor. In some embodiments, catheter 102 includes a filter with micro-pores which provides back pressure to the delivered steam, thereby pressurizing the steam. The predetermined size of micro-pores in the filter determine the backpressure and hence the temperature of the steam being generated. In some embodiments, the system further comprises a foot pedal in data communication with controller 5710, a switch on catheter 102, or a switch on the controller 102, for controlling vapor flow. In various embodiments, the switch to control the vapor flow is positioned on the generator or the catheter handle.

In one embodiment, a user interface 5820 included with the generator system 5800 allows a physician to define device, organ, and condition which in turn creates default settings for temperature, cycling, volume (sounds), and standard RF settings. In one embodiment, these defaults can be further modified by the physician. The user interface also includes standard displays of all key variables, along with warnings if values exceed or go below certain levels. In embodiments, user interface 220 includes a touch-screen display 221.

The ablation device also includes safety mechanisms to prevent users from being burned while manipulating the catheter, including insulation, and optionally, cool air flush, cool water flush, and alarms/tones to indicate start and stop of treatment.

Referring now to FIG. 2, illustrating generator 200, fluid such as saline is stored in a reservoir 223. A pump is positioned in fluid communication with the reservoir. In some embodiments, the pump is a syringe pump 222. The reservoir and pump arc positioned within an enclosure or housing 224. The housing 224 can be made from a partially transparent material. Further, pump or syringe 222 is in communication with fluid line 130 of catheter 102 through a fluid port 225. Delivery of fluid through port 225 into catheter 102 is controlled via controller 110 by a treating physician using interface 220 provided on generator 200 to activate the pump. Interface 210 may be a touch-screen interface 221. Fluid port 225 can also be positioned toward a side or front surface of generator 200, allowing the user/physician to view and control through display 220 while attaching the catheter 102. The generator 200 includes an RF catheter connection port 212 for connecting to an RF cable 128 of the catheter 102. Generator 200 includes a controller 110 having at least one processor in data communication with interface 220 and configured to control the delivery of RF current, via the RF connection port 212, and the delivery of fluid, via the fluid port 225, to the catheter, the pump and catheter fluid port 212 that is in fluid communication with the pump. A button 218 is provided on the front panel of generator 200, to manually activate ablation of fluid using RF ablation methods. In some embodiments, the front panel of generator 200 also includes at least one USB port 226. Port 226 can be used to connect a power supply to operate generator 200, or to charge rechargeable batteries within generator 200.

With reference to FIG. 3, illustrating a side view of catheter 102, an RF cable 128 is attached to a proximal end of catheter 102, which is configured to interface with RF catheter port 212 of FIG. 2. Additionally, a saline fluid line (or cable) 130 is also attached adjacent to cable 128 to the proximal end of catheter 102, which is configured to interface with the syringe pump through fluid port 225 of FIG. 2. Handle 116 provides slider mechanisms to control the movement of an outer sheath, which when pulled back enables deployment (expansion) of the first and second positioning elements 104, 108, and optionally to control the positioning of first positioning element 104.

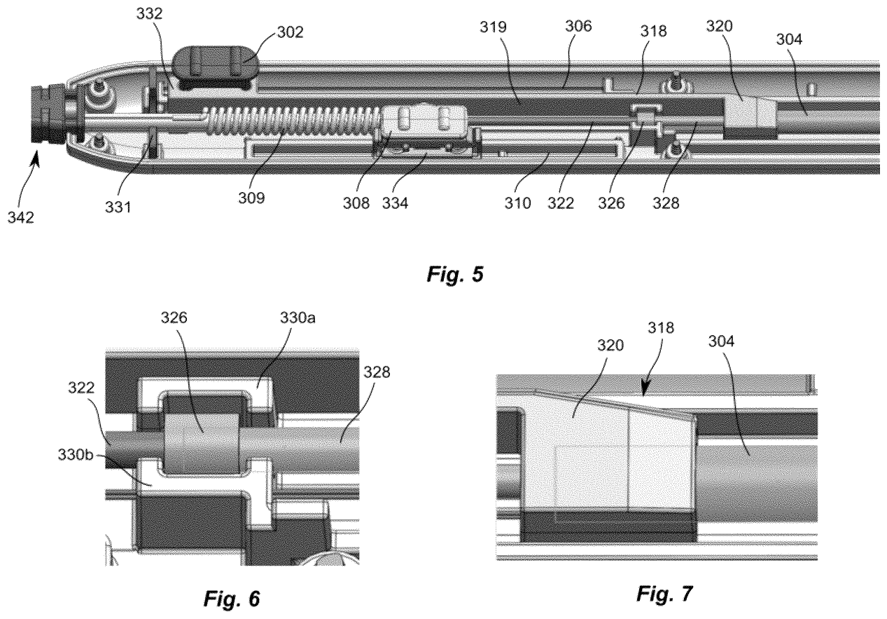

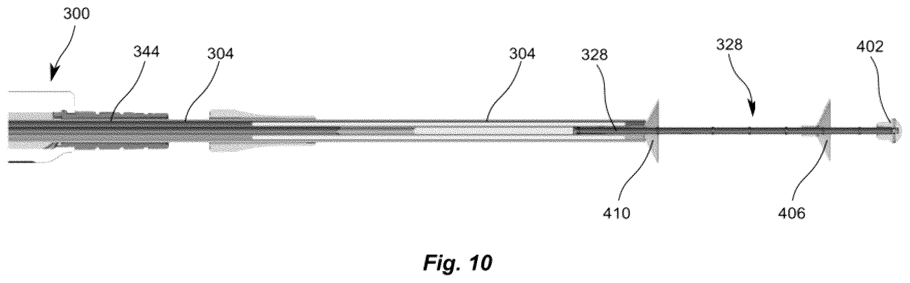

FIG. 4 illustrates a view of the top surface of handle 300 of catheter 102. FIGS. 5-7 illustrate a view of the internal components of handle 300. FIG. 10 illustrates a cross-section view along a longitudinal axis of catheter device 100 extending from handle 300 toward a distal olive-shaped tip 302. Referring simultaneously to FIGS. 4-7 and 10, handle 300 is configured in an elongated flat design with its top surface holding a first slider 302 to control the movement of an outer sheath 304. Slider 302 is configured to slide smoothly through a pushing action by the user, within a channel 306 of a fixed first length. A second slider 308 is configured parallel to first slider 302 to control the positioning of a distal positioning element 406. Second slider 308 is configured to slide smoothly through a pushing action by the user, within a channel 310 of a fixed second length. A series of visual markings 312 are embossed or otherwise illustrated alongside the length of channel 310. Markings 312 enable the user to visualize a length for adjusting the position of second slider 308 corresponding to the size of the uterine cavity. Additional markings are embedded or illustrated on the top surface of handle 300 to indicate the functions of first and second sliders 302, 308. For example, a marking 314 at a proximal side of channel 306 indicates that movement of slider 302 in the direction of marking 314 withdraws or pulls back the outer sheath 304 of the catheter to deploy the positioning elements. Similarly, a marking 316 at a distal side of channel 306 indicates movement of slider in the distal direction to cover and compress the positioning elements with the outer sheath 304. Movement of slider 302 till the most distal position within channel 306 would move outer sheath 304 up to the distal olive-shaped tip 402, covering and compressing the two positioning elements. Marking 319, at a distal side of channel 310, indicates to the user that second slicer 308 controls the positioning of the distal end or first positioning element within the uterine cavity.

FIGS. 5 and 7 further illustrate an attachment 318 of first slider 302 to outer sheath 304. In embodiments, attachment 318 is an elongated solid component. A proximal end of attachment 318 provides a platform 332 to fixedly attach first slider 302. Attachment 318 extends distally parallel to channel 306 and ends in a distal portion 320 that is flared in proportion to the elongated middle portion 319 of attachment 318. Outer sheath 304 is attached to the distal portion 320 of attachment 318 using an adhesive, in some embodiments.

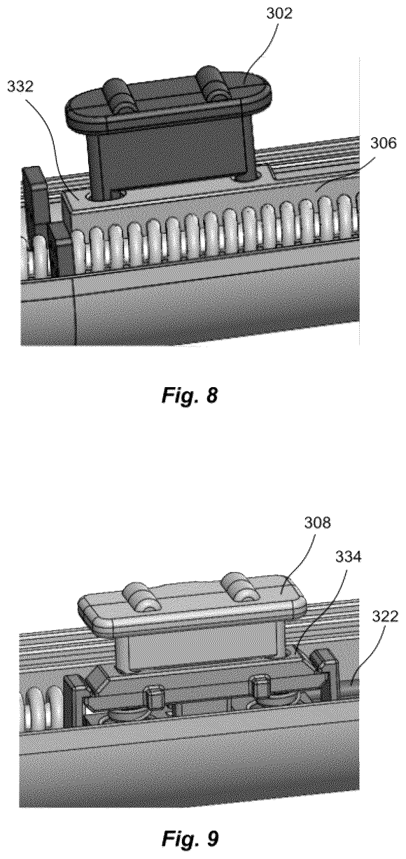

FIGS. 5 and 6 further illustrate an attachment 322 of slider 308 to a shaft 328 of the catheter. Attachment 322 is an elongated solid component. A proximal end of attachment 322 provides a platform 334 to fixedly attach second slider 308. Attachment 322 extends parallel to channel 310 and ends in a distal portion that is configured with a collar 326. Collar 326 is positioned between a first retaining wall 330a and a second retaining wall 330b, each positioned on either side of collar 326, to stabilize the proximal shaft 328 within the handle 300. FIG. 8 illustrates a view of first slider 302 attached to platform 332 at the proximal end of attachment 306. FIG. 9 illustrates a view of second slider 308 attached to proximal end 334 of attachment 322.

Referring to FIGS. 4 and 5, a cord grip 342 on a proximal end, and a cord grip 344 on a distal end of handle 300, strengthen the attachment and flexibility of handle 300. Cord grip 342 provides protection and stability to the RF cable 128 and fluid cable 130, referring to FIG. 3, extending from the proximal end of the handle 300, and cord grip 344 provides protection and stability to the outer sheath 304 and catheter shaft 328 extending from the distal end of the handle 300. In some embodiments, a board 331 is positioned within the handle, proximate a proximal end of the handle, and is configured to secure and manage the various components and connections within the handle. In embodiments, the handle includes an RF wire coil 309 for delivering electrical current to the at least one electrode within the heating chamber of the catheter.

The multiple embodiments of handle mechanism include systems for deployment and retraction of positioning elements, which can be achieved in different ways such as and not limited to buttons, push/pull on distal or proximal end, slide button in a track, rotation with push/pull. The internal components of the embodiments of the handle mechanism are made generally using stainless steel. The external components of the embodiments of the handle mechanism are made generally using a combination of ABS, plastics, rigid polymers, and elastomeric polymers, among other material. The various embodiments also provide for strain relief for the back end for the fluid tube and the electrical cable, and strain relief on the front end to provide support for the catheter segment. In various embodiments, the handles described in the present specification have lengths ranging from 3 inches to 24 inches and diameters or widths ranging from ¼ inch to 5 inches.

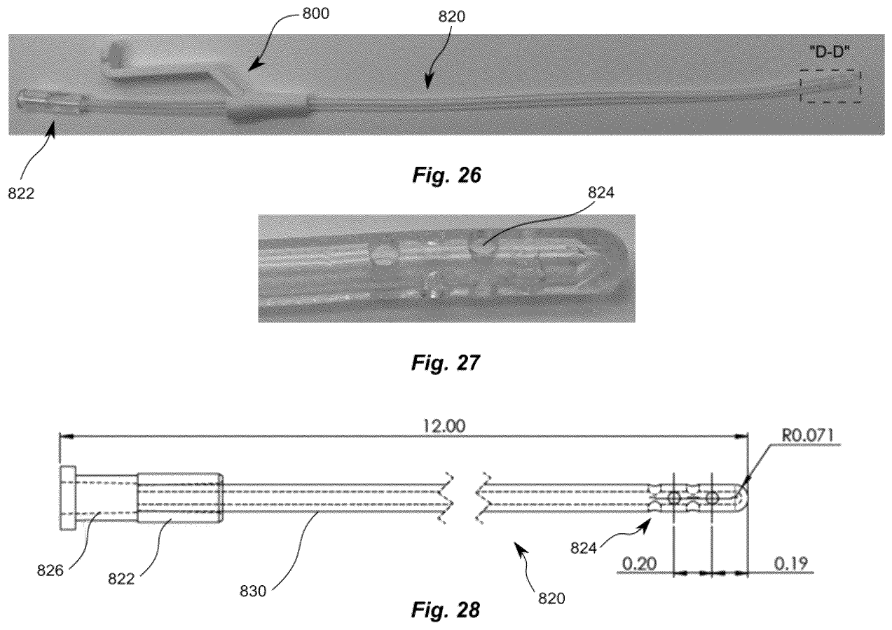

To prepare the catheter, a physician sets a position of second slider 308 within channel 310, according to the measured size of uterine cavity. Moving slider 308 to the measured uterine cavity size (FIG. 4 shows slider 308 positioned at a size ‘9’) causes distal positioning element 406, olive tip 402 (referring to FIG. 10) at the distal end of the catheter, and an internal member within outer sheath 404, to which both (the distal positioning element and the olive tip) are attached, to move. Slider 308 distance is set at a value that less than a measured uterine cavity size. The uterine cavity size is measured using conventional techniques. In some embodiments, the physician measures the uterine cavity with a conventional uterine sound technique. In embodiments, the uterine cavity is measured using an aspiration catheter with insertable stylet, as discussed with reference to FIG. 26-28. The units of measurement are expressed herein in centimeters (cm) which typically range from 3 to 6 cm. Using the uterine size obtained, the physician sets the degree of expansion for the positioning elements of catheter, using slider 308. The distance set using slider 308 is slightly less than the measured uterine cavity size, to ensure that there is enough space distal to the distal end of the catheter (olive tip 402) for ablative agent to circulate. In an illustrative example, if the slider is set to “5” (indicative of the uterine cavity size), a distal assembly 408 (comprising the distal positioning element 406 and the olive tip 402) moves such that a distance from a distal surface of a proximal positioning element to the end of the olive tip is slightly less than 5 cm, such as for example 4 cm. In embodiments, slider 308 can be set from “3” to “9”. The proximal positioning element 410 is stationary. Once second slider 308 is moved (causing distal assembly 408 to move), slider 308 is locked, thereby locking distal assembly 408 in place.

In some embodiments, first slider 302 is of a first shape, such as for example an oval shape, and a first color, and second slider 304 is of a second shape, such as a square or a rectangular shape, and a second color, enabling easy distinction between the two sliders that serve different purposes. Button slider support features are added on an inner side proximal to a bottom surface of handle 300. Buttons of sliders 302 and 308 are press fitted on the platforms/proximal portions 332 and 334 of the sliding mechanisms, using a poka yoke button design.

Once distal hood slider 308 is locked, a test shot is performed to ensure vapor is being properly generated. Using outer sheath first slider 302, the outer sheath is advanced distally along the catheter shaft 328 to cover and compress positioning elements 406 and 410. The outer sheath slides forward to eventually meet olive-shaped tip 402 so that the sheath 304 and olive tip 402 create an atraumatic surface. The atraumatic surface ensures there are no openings in the distal end of the catheter that would catch on tissue as the catheter is inserted into a body cavity. The catheter is then inserted into the patient, outer sheath slider 302 is slid back to uncompress positioning elements 306 and 310, and a cervical stabilizer 118 is slid into position.

Referring back to FIG. 3, a cervical stabilizer 118 is positioned coaxially about the catheter sheath 119, located on a proximal side to proximal positioning element 108, and comprises a conical-shaped element 120 having a central lumen through which the catheter shaft extends. The conical-shaped element 120 is attached to a slant portion 122 extending proximally and which is attached to a parallel portion 124 that further extends proximally and, in turn, is attached to a perpendicular portion 126, where each portion 122, 124 and 126 of cervical stabilizer 118 is described relative to a central longitudinal axis of the catheter shaft. Cervical stabilizer 118 ensures stability and tactile control of the distal portion of the catheter. A physician holds stabilizer 118 during insertion of the catheter inside cervix, to support its placement. Stabilizer 118 does not actively seal the cervix, where sealing is not required as a result of the small amount of fluid that is dissipated into the tissue during the ablation process. Stabilizer 118 can be positioned depending on anatomy of the patient, to stabilize the catheter.

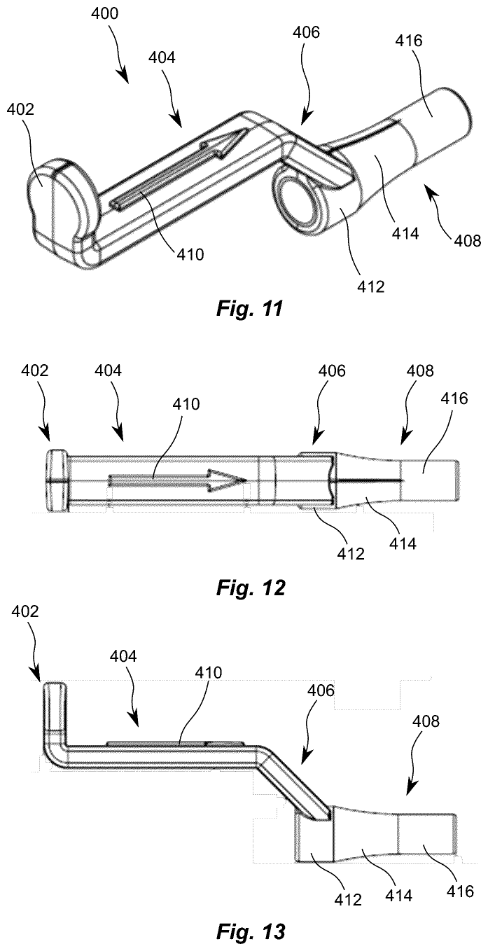

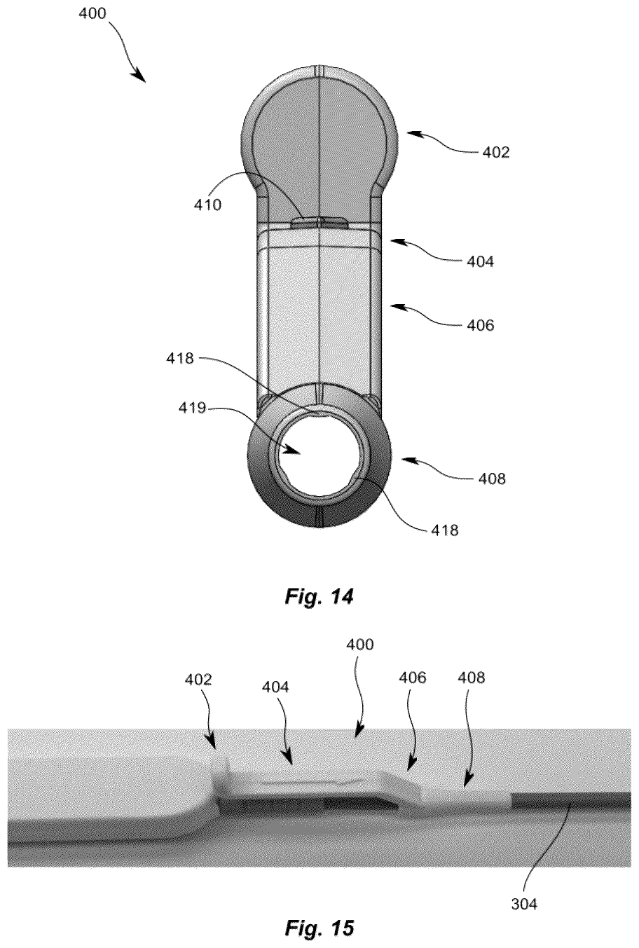

FIG. 11 illustrates a perspective view of an embodiment of cervical stabilizer 400, in accordance with the present specification. FIG. 12 illustrates a top view of cervical stabilizer 400, in accordance with the present specification. FIG. 13 illustrates a side elevation view of an exemplary embodiment of cervical stabilizer 400, in accordance with the present specification. FIG. 14 illustrates a three-rib configuration in an embodiment of cervical stabilizer 400, in accordance with the present specification. FIG. 15 illustrates a photograph of a cervical stabilizer 400, in accordance with some embodiments of the present specification.

Referring simultaneously to FIGS. 11-15, stabilizer 400 is described in four sections—a first most proximal section 402; a second section 404 attached to and perpendicular to first section 402 and extending in a distal direction relative to the first section 402; a third section 406 attached to and at an angled orientation relative to second section 404, and extending in a distal direction relative to the second section 402; and a fourth section 408 attached to the third section 406 and oriented parallel to second section 404 and extending in a distal direction relative to the third section 406, wherein a distal end of the third section 406 is attached to a side of the fourth section 408 proximate a proximal end of the fourth section 408. Together, sections 402, 404, 406, and 408 form cervical stabilizer 400. Proximal section 402 has a flat circular shape, with a thickness of approximately 0.08 inches to 0.50 inches (preferably 0.160 inches) and a radius of approximately 0.215 inches. First section 402 provides a handle to hold cervical stabilizer 400 during insertion of catheter system (and a uterine aspiration catheter) inside the cervix. A height of first section 402 up to a level of a top surface of section portion 404 is approximately 0.444 inches. Second section 404 is continually attached to first section 402 at a smooth, curved joint with a radius of approximately 0.173 inches. Second section 404 has a flat, elongated rectangular shape, with a thickness of approximately 0.08 inches to 0.50 inches (preferably 0.160 inches), and a length of 0.50 inches to 2 inches, and preferably 1.26 inches. In some embodiments, at a distance of approximately 0.286 inches, an arrow 410 is embossed on a top surface of second section 404, which extends to point toward a distal direction for a length of approximately 0.973 inches. In some embodiments, thickness of the embossed arrow 410 is approximately 0.029 inches above the surface of second section 404. Third section 406 is continually attached to second section 404 at an angle of approximately 135° measured from the bottom surface of second section 404. Third section 406 provides a leverage to maneuver cervical stabilizer 400 by holding from outside the uterine cavity. The lever is a pusher lever used by the physician to push stabilizer 400 forward. Distal end of third section 406 slopes downwards to fixedly attach to a side of a proximal portion 412 of fourth section 408.

Fourth section 408 further includes three portions-proximal cylindrical first portion 412 that extends distally into a cone-shaped second portion 414, which extends further distally into a distal cylindrical third portion 416. The total length of fourth section 408 is approximately 1.137 inches. First portion 412 has a length of approximately 0.271 inches and an outer diameter of approximately 0.394 inches. Second portion 414 has a proximal diameter to match the outer diameter of first portion 412. Second portion 414 narrows towards the distal end where it has a diameter in a range of approximately 0.280 inches. The distal end of second portion 414 is continually attached to the third portion 416 which is cylindrical in shape. An outer diameter of third portion 416 is approximately 0.28 inches. First, second, and third portions 412, 414, 416, are configured to be hollow to accommodate passage of outer sheath of ablation catheter or outer sheath of aspiration catheter, where the catheter sheath passes through the length of fourth section 408. In embodiments, a plurality of ribs 418 are positioned within third portion 416 of fourth section 408. Three ribs 418 are shown in the illustrated embodiment. Each rib 418 extends inwardly from an inner circumference of third portion 416 proximally for a length of approximately 0.089 inches into a space 419 within the fourth section 408. Each rib 418 is spaced equally around the circumference of internal circular surface of proximal end of third portion 416. Gaps between the ribs ensure that no seal is created. Ribs 418 provide a friction fit to the catheter sheath (or aspiration catheter) passing through fourth section 408 of cervical stabilizer 400. Ribs 418 slide up and down on the catheter sheath when stabilizer 400 is pushed/pulled by physician, and holds stabilizer 400 to the catheter sheath when released by the physician.

In embodiments, cervical stabilizer 400 is made from a rigid, non-compliant material, such as for example PP homopolymer profax PF511. In some embodiments, cervical stabilizer 400 is of a bright white color to enable easy spotting and visibility of the device for holding by the physician. In embodiments, stabilizer 400 used with the treatment catheter and with the aspiration catheter are identically designed and provide the same function.

FIGS. 16-18 illustrate a cross-section view, with selected expanded views thereof, of a catheter lumen 500 including expanded views of a proximal portion 530 of an electrode assembly core 532 and a proximal end 534 of a distal shaft 536, in accordance with some embodiments of the present specification. A portion of the catheter lumen extending between a cervical stabilizer component 538 and extending up to a proximal end of proximal positioning element 510 comprises an internal heating chamber 540. The internal heating chamber is encapsulated with an insulation coating 542. In embodiments, the insulation coating includes materials such as polyimide, aerogel, AcroZero®, and silicone, as further shown and described with respect to FIG. 25. Insulation coating 542 is configured to prevent tissue damage or burns by limiting the transfer of heat from the internal heating chamber to the uterine cavity. The heating chamber 540 is positioned within a lumen 548 of the catheter. Inside the heating chamber 540 is positioned at least one electrode or an array of electrodes 539 positioned coaxially around, or wrapped about, an electrode core 532. The electrode core 532 is configured to prevent turbulent flow and maximize fluid contact with the at least one electrode 539. In embodiments, the electrode core is a solid material and configured in a cylindrical shape. In some embodiments, the electrode core is comprised of polyether ether ketone (PEEK). In some embodiments, the electrode core 532 is also an electrode configured to convert the fluid to vapor. In some embodiments, the heating chamber 540 comprises both at least one electrode 539 positioned circumferentially around an electrode core 532 wherein the electrode core 532 is also an electrode. In some embodiments, the heating chamber 540 comprises only an electrode core 532 wherein the electrode core 532 is an electrode. The electrode core 532 serves to improve vapor generation by reducing the volume for saline to flow through while passing over the electrodes. As the fluid travels in the lumen 548 and approaches the electrode core 532, the cross-sectional area of the fluid pathway decreases, forcing the fluid to come into contact with the electrodes 539 positioned around the electrode core 532. In some embodiments, the cross-sectional area of the fluid pathway ranges from 3 mm²-30 mm² before the heating chamber 540 and from 1 mm²-20 mm² within the heating chamber 540, with a proportional reduction in the cross-sectional area of the fluid pathway ranging from 10% to 90% from pre-heating chamber to within the heating chamber. In embodiments, the proximal end of the electrode core 532 has a tapered configuration with an angle of a ramp ranging from 5 degrees to 85 degrees, starting from the proximal tip 533 of the electrode core 532 to a tapered end 535 of a ramped section 537 of the electrode core 532. In embodiments, this ramped section 537 improves the uniformity of distribution of the fluid over the electrodes 539 of the heating chamber 540 by at least 10%, wherein uniformity of distribution is quantified as volume of saline over the surface area of each quadrant of the electrode. In embodiments, the improvement of the uniformity of distribution of the fluid over the electrodes by including an electrode core with a tapered proximal end results in improvement of the quality of steam from <25% to >40%, or a 25% improvement, relative to vapor generation absent an electrode core. In embodiments, the electrodes 539 and core 532 are separated from thermally conductive elements of the catheter by segments 544 of catheter which are electrically non-conductive. Each electrode 539 and the electrode core 532 are configured with a tapered edge at their proximal ends 530. In some embodiments, a length of the tapered proximal end of the electrode core is in a range of 1 mm to 15 mm, and more preferably 2 mm to 10 mm. The tapered configuration of electrode(s) 539 and electrode core 532 prevents electrode shortening and ensures that flow of ablation fluid within lumen 538, flowing over the tapered ends, is rendered less turbulent, relative to that offered by non-tapered configurations. As an ablation fluid approaches tapered ends indicating the start of the electrodes 539 and electrode core 532, the fluid's flow is rendered laminar thus resulting in less turbulence and more consistent contact across the surface of the electrodes 539.

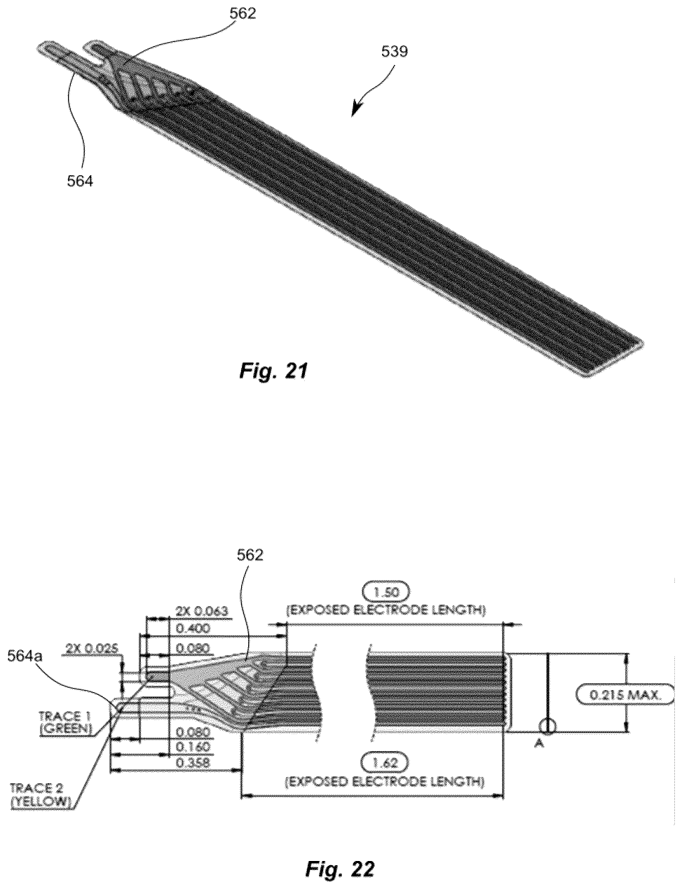

FIGS. 21 and 22 illustrates a first array of electrodes 562 and a second array of electrodes 564 for positioning around an electrode core within a flexible heating chamber, in accordance with some embodiments of the present specification. Proximal sides of both arrays of electrodes 562 and 564 have a tapered configuration, and further, each tapered edge of electrodes 562 and of electrodes 564 are offset from each other to prevent electrode shorting once positioned inside the heating chamber. FIG. 22 illustrates a close-up view of the tapered proximal edge configurations of arrays of electrodes 562 and 564, in accordance with embodiments of the present specification. A first tapered proximal edge 562a corresponds to electrodes 562 and a second tapered proximal edge 564a corresponds to electrodes 564. Arrays of electrodes 562 are interlaced with arrays of electrodes 564, such that a width 566 of both arrays 562 and 564 together is approximately 0.215 inches. A length 562b of exposed electrodes of arrays of electrodes 562 is approximately 1.5 inches, whereas a length 564b of exposed electrodes of arrays of electrodes 564 is approximately 1.62 inches, resulting in an offset at the proximal sides of arrays of electrodes 562 and 564. Further, length of first tapered proximal edge 562a is shorter than length of second tapered proximal edge 564a. In some embodiments, length of edge 562a is approximately 0.063 inches and that of edge 564a is approximately 0.358 inches. The offset solder pads configured at the offset proximal edges 562a and 564a of the arrays of electrodes 562 and 564 prevents shorting when the two arrays 562 and 564 are wrapped together inside the heating chamber. Therefore, tapered edge 530 configuration of electrodes 532 overcomes a prior design limitation wherein the proximal portion of the electrodes have a straight edge resulting in the possibility that the soldered joints of the proximal edges could touch and short.

Referring again to FIG. 15-20, a distal side of electrode core 532 is positioned proximate a proximal side 534 of a distal shaft 536 that extends up to and is attached to an olive-tip 502 at the distal end of the catheter. In embodiments, proximal side 534 of distal shaft 536 is shaped like a “crown” (or like the tip of a rook) to facilitate the flow of fluid converted to vapor by electrode 532, into the distal shaft 536. The crown shape prevents the entirety of proximal end of the distal shaft from contacting the distal end of the electrode core and blocking the flow of vapor. An uneven proximal end 534 shaped in the form of a crown provides gaps 531 for the vapor 561 to flow through in the event that the crown-shaped proximal side 534 touches the distal side of electrode core 532, thereby improving the flow of vapor.

In embodiments, olive tip 502 is configured to have at least one vapor delivery port 550 positioned on a side of the olive tip 502 and proximate the distal end dome-shaped tip of the atraumatic olive tip 6302. In embodiments, the olive tip includes a range of 1 to 20 vapor ports. FIGS. 19 and 20 illustrate a olive-shaped distal tip 502 with a plurality of ports 550. A distal end 554 of catheter shaft 504 terminates within tip 502 prior to ports 550. The embodiment includes a total eight ports 550, some visible with others not shown on the opposite side. All ports are configured through the body of olive tip 502 and may be positioned at equal angles from each other within the circumference of distal tip 502. Each port extends outwards radially from a center 552 axis of the catheter shaft, toward the outer circumference of olive tip 502. In embodiments, 1-50 delivery ports, and preferably 12-15 vapor ports 503, are positioned along the length of the catheter shaft 504 between proximal positioning element 510 and distal positioning element 506. Once deployed within the uterine cavity, the vapor formed within the heating chamber is released via the ports in the catheter shaft and in the olive tip and extends upwards into the uterus and flows around the surface of the uterus distally to proximally. First-sized ports proximally along the catheter shaft 504 and relatively larger second-sized ports distally on shaft 504 result in more vapor being emitted distally. As the distal assembly (comprising distal shaft 536, distal positioning element 506 and olive tip 502) is moved and locked, some vapor ports may become blocked by virtue of remaining positioned within the outer sheath of the catheter at a proximal side of proximal positioning element 510. In said scenario, configuration of first-sized ports positioned at the proximal side of shaft 504 is an advantage. The blocked ports are of a relatively smaller size, thereby still allowing sufficient vapor to be released in the uterus through the distal ports of relatively larger sizes.

In embodiments, the catheter is made of or covered with insulated coating 542 to prevent the escape of ablative energy from the catheter body to avoid possibility of burning in the likelihood where the catheter shaft comes in contact with the tissue surface inside the uterus, as the fluid flowing over electrodes 539 is heated. Therefore, insulation coating 532 lowers the surface temperature of the catheter shaft configured to be positioned within the body. In embodiments, the insulation coating 542 is positioned coaxially between an inner wall 545 (or proximal and distal inner shafts) of the catheter shaft and an outer wall 541 of the catheter shaft.

FIG. 25 illustrates composition that, in embodiments of the disclosure, forms one insulation layer 600 used in some embodiments of the present specification. An innermost material 602 comprises a release lining, a second material 604 covering innermost material 602 includes an adhesive coating, a third material 606 layered over second material 604 comprises a flame and thermal barrier (for example aerogel), a fourth material 608 again provides an adhesive coating over the outer side of third layer 606, and a fifth outermost material 610 includes a polyimide surface. In some embodiments, at least four layers of insulation, comprising each material of the insulation materials described in FIG. 25, is wrapped together to comprise the insulation coating 542 of FIG. 16. Table 1, shown below, demonstrates the time needed to cause a burn when the surface temperature of a catheter shaft is maintained at a certain temperature. Table 1 lists different temperatures, ranging from 45° C. to 60° C. Table 1 also lists the time at each temperature to cause a second degree burn that does not cause irreversible damage. Table 1 further lists the time at each temperature to cause a third degree burn that can result in an injury. Table 1 illustrates, for example, a surface temperature of the catheter shaft will have to be at 51° C. for at least 2 minutes (min) to cause a second degree burn, and for at least 4.2 min to result in a third degree burn.

TABLE 1

| | | |
|---|---|---|
| | 2^{nd} Degree Burn | 3rd Degree Burn |
| Temperature | (No irreversible damage) | (full thickness injury) |

| | | | | |
|---|---|---|---|---|
| 45° C. | 2 | hours | 3 | hours |
| 47° C. | 20 | minutes | 45 | minutes |
| 48° C. | 15 | minutes | 20 | minutes |
| 49° C. | 8 | minutes | 10 | minutes |
| 51° C. | 2 | minutes | 4.2 | minutes |
| 55° C. | 17 | seconds | 30 | seconds |
| 60° C. | 3 | seconds | 5 | seconds |

FIG. 25A illustrates another table to demonstrate the results of an insulation test performed with insulation layers 600 in accordance with the embodiments of the present specification. In FIG. 25A, the X-axis lists the number of insulation layers (wraps) used to coat the outer surface of the catheter shaft, and Y-axis lists the different temperatures (in° C.) achieved on the outer surface of the catheter shaft during an ablation procedure corresponding to the different number of layers. Outer surface temperature of a catheter shaft comprising four layers, in accordance with the present specification, reaches a maximum of 47.3° C., which according to Table 1, would require approximately 45 minutes to cause an irreversible burn injury. The actual time required to complete the ablation process within the uterine cavity is considerably less than 45 minutes.

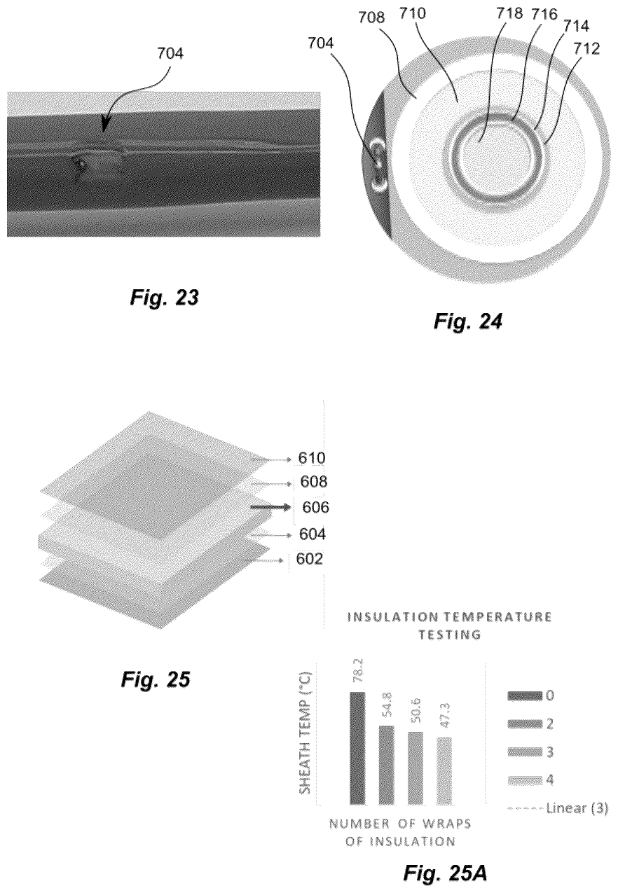

Referring now to FIGS. 23 and 24, in some embodiments, a thermocouple is integrated into the outer layers of the catheter to measure the temperature of the outermost layer of the catheter. In embodiments, the thermocouple is housed within an outer lumen of the catheter shaft configured with multiple lumens. In some embodiments, the thermocouple is housed between the outermost wall and the next to the outermost walls of the catheter, and positioned along the catheter proximate the heating chamber/electrodes. The thermocouple is configured to measure the temperature of the outermost layer of the catheter itself, and not a temperature of the ablative agent or uterine cavity as is typical in the prior art. In some embodiments, the system is configured to measure and confirm the temperature of the outermost layer of the catheter is 51° C. or less for at least 30 seconds. The thermocouple tip is inset into the catheter's outer surface. The ablation system is configured to shut down if catheter shaft temperature rises above a threshold temperature, as measured with the thermocouple. The threshold temperature is a temperature that can damage the patient by causing an injury.

FIGS. 23 and 24 illustrates a cross-section view 702 of the catheter shaft along an axis perpendicular to the longitudinal axis of the shaft, and a position of a thermocouple 704 is shown in FIG. 23 where thermocouple 704 is positioned within the outer-most lumen of a multi-lumen outer shaft of catheter. FIG. 24 illustrates layers of the catheter shaft comprising: an outer wall 708, an insulation layer 710 (equivalent to set of layers 600), a proximal shaft 712 and distal catheter shaft 714 (inner walls), an electrode array 716, and an electrode core 718.

An aspiration catheter is used to clean the field of ablation within the uterine cavity, before and/or after a conventional hysteroscopy. Figure FIGS. 26-28 illustrate a uterine aspiration catheter 820 in accordance with embodiments of the present specification. A cervical stabilizer 800 is positioned coaxially over the uterine aspiration catheter 820 and is configured to stabilize the uterine aspiration catheter 820 during aspiration, similarly to stabilizing the ablation catheter during ablation. A proximal end of uterine aspiration catheter 820 is fitted with a luer connection 822 and a distal portion of catheter assembly 820 includes one or more aspiration ports 824. In embodiments, uterine aspiration catheter 820 is flexible and includes markings (not shown) along its length. To measure or sound the uterine cavity, in embodiments, a rigid elongated device, such as a stylet (not shown), is inserted into the lumen of the 822 of the uterine aspiration catheter once it has been positioned in the uterus. The markings 821 are then read by the physician and indicate to the physician the size of the uterine cavity. In some embodiments, the stylet includes a handle. Therefore, in embodiments of the present specification, the uterine aspiration catheter 820 is configured to function as both an aspiration catheter and uterine cavity measuring device.

FIG. 28 illustrates a side elevation view of aspiration catheter 820. Referring simultaneously to FIGS. 26-28, Luer connection 822 includes a female luer connector 826 which is configured to secure attachment of aspiration catheter assembly to a syringe. In embodiments, luer connector 826 is configured preferably to interface with a 20 cc syringe for input of fluid such as saline. Tubing 830 is made from a flexible material, such as, for example a flexible polyurethane braiding. In other embodiments, material for tubing 830 is selected from spectrum plastic group P/N: FA2775-100P or equivalent. An internal diameter of tubing 830 ranges from 0.05 inches to 0.071 inches, and external diameter is approximately 0.142 inches. A length of uterine aspiration catheter assembly 820 is, in an embodiment, approximately 12 inches. In some embodiments, at least two ports 824 are aligned along a straight line parallel to a longitudinal axis of tubing 830, at a distance of approximately 0.20 inches. At least two more ports 824 are aligned along a straight line and offset from the first pair of ports 824 axially by an angle of approximately 90°, as well as linearly by approximately 0.1 inches. The most distal port(s) 824 has a center at a distance of approximately 0.19 inches from the distal tip of tubing 830. Each port of ports 824 has a radius of approximately 0.071 inches.

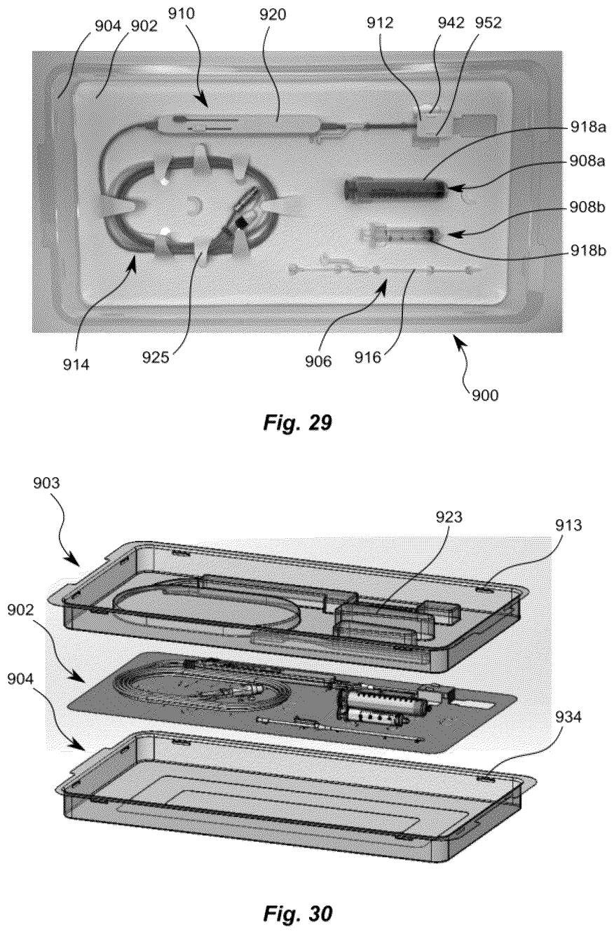

FIG. 29 illustrates multiple components of an ablation system of the present specification, packaged together in a kit 900. FIG. 30 illustrates another view of the packaging elements of kit 900, in accordance with some embodiments of the present specification. Referring simultaneously to FIGS. 29 and 30, a middle layer or substrate of the kit, comprising a card 902, is configured to house multiple components of the ablation system, including the ablation catheter, syringes, and uterine aspiration catheter. In embodiments, card 902 comprises a plurality of spacings for placement and securing of the components of the ablation system. In some embodiments, card 902 includes first spacing 916, second spacing 918a, third spacing 918b, fourth spacing 920, and fifth spacing 924 for an aspirator catheter 906, syringes 908a and 908b, an ablation catheter 910, and cables 914 of the ablation catheter respectively. As discussed previously, the catheter must be shipped with the positioning elements in their expanded configuration to prevent misshaping of the positioning elements. In embodiments, the card 902 includes at least one first portion or cover 912 configured to cover and protect a component of the ablation system. In embodiments, cover 912 comprises a cut-out flap configured fold away from a flat plane of the card 902 and cover the distal end of the ablation catheter 910 to protect the positioning elements during shipment. In some embodiments, card 902 comprises second and third portions or cover wings 942, 952 configured to fold away from the flat plane of the card 902 and engage with the at least one first cover 912 to form an enclosure over a component of the ablation catheter 910. Card 902 (die card) is placed within a bottom layer, base, or rectangular tray 904. Tray 904 is a thermoformed tray. A top layer, or molded tray lid 903, is used to cover the top surface of components placed within card 902. In embodiments, molded tray lid 903 is a thermoformed rectangular tray comprising a plurality of recessed cavities 923 that are recessed according to shapes of stored components within card 902. In embodiments, lid 903 includes first latching components 913 configured to engage with second latching components 934 of tray 904 to securely lock the lid 903 with the tray 904 with the card 902 in between.

Cables 914 of ablation catheter 910 are coiled and positioned and clipped within card 972 with adjustable open-ended clips 925 that are cut into card 902. Embodiments of ablation catheter system are packaged and shipped with two positioning elements being unsheathed such that they are in an expanded configuration, which is the configuration assumed by the positioning elements during deployment within the uterine cavity. The positioning elements are package and shipped unsheathed since, in compressed form, there is a possibility that, when the positioning elements are subsequently unsheathed, they might not expand as a result of a sterilization process which, if done on the positioning elements in their compressed configurations, could cause them to remain compressed. Card 902 and molded tray lid 903 secure the components of the ablation device for transportation and storage. Kit 900 is, in some embodiments, further packaged and scaled inside a Tyvek pouch to ensure a sterile barrier. The pouch can be placed within a box, and can be shipped in a bigger box with other boxes containing a pouch each.

In embodiments, components of kit 900 are removed and calibrated outside a patient, before a procedure, and while in communication with a generator (200 of FIG. 2). In embodiments, slider to control the positioning of the outer sheath of the catheter is pulled all the way back so that the outer sheath is not positioned over any of the positioning elements. Additionally, the slider to control the distal element comprising the distal positioning element and the olive-shaped tip at the distal end of the catheter, is positioned and locked for the maximum uterine cavity size. In one embodiment, the slider that controls the distal element is positioned at number ‘5’ corresponding to a uterine cavity size of 5 cm, resulting in locking of the device so that the distance from the proximal positioning element to the olive-shaped distal tip is approximately 4 cm. The uterine cavity size is an indication of the measurement from fundus to internal cervical os.

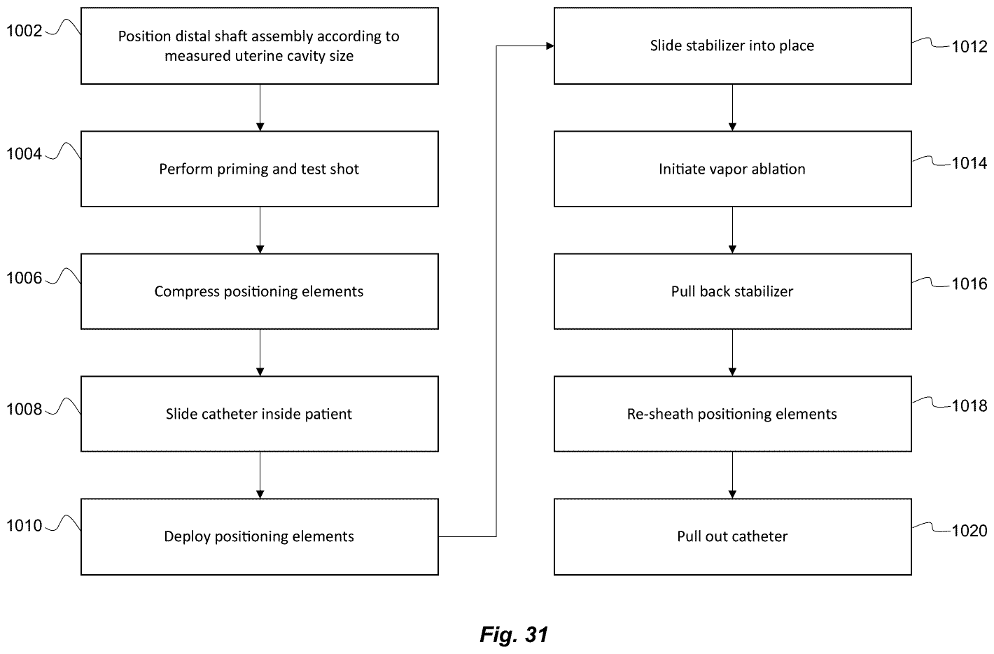

FIG. 31 is a flow chart illustrating an exemplary method for preparing and using the ablation catheter device or system described in accordance with the various embodiments of the present specification. At step 1000, a physician positions the distal positioning element slider according to a measured uterine cavity size. Moving the distal positioning element slider to the measured uterine cavity size causes the distal positioning element, olive tip at the distal tip of catheter device, and an internal member within the outer sheath, to which both (the distal positioning element and the olive tip) are attached, to move. The slider distance is set at a value that is less than a measured uterine cavity size. A distance between the proximal positioning element and the distal tip of the catheter is approximately 1 cm less than the measured size of the uterine cavity, thus ensuring that the ablation mechanism placed within the body cavity does not contact the body tissue. The uterine cavity size is measured using conventional techniques. In some embodiments, the physician measures the uterine cavity with a conventional uterine sound technique. The units of measurement are expressed herein in cm which typically range from 3 to 6 cm. Using the uterine size obtained, the physician sets the degree of expansion for the positioning elements of the catheter device, with the slider. A proximal positioning element is stationary. Once the slider is moved (causing the distal assembly to move), the slider is locked, thereby locking the distal assembly in place.

In some cases, the physician uses the aspiration catheter after the measurement of the uterine cavity to remove water/fluid from the uterine cavity before inserting the catheter to avoid problems of water and steam mixing during ablation.

At step 1004, priming and a test shot are performed wherein fluid is passed through the catheter and is heated by electrodes to generate ablation vapor. The generated vapor exits through the infusion ports, including the ones between the positioning elements, the ports distal to the distal positioning element, and the port(s) on the (olive-shaped) distal tip. The vapor exit is visually verified by the physician. Once a puff of vapor is passed, the ablation device is considered to be ready to be used for treatment. The steam is generated by pressing a button on the generator interface or by pressing on a foot pedal. In some embodiments, the vapor is delivered for a period of time that is less than five minutes, and preferably for a period of time within a range of 0 to 120 seconds, and more preferably 90 seconds. The vapor delivered at this stage is for the purpose of priming and preparing the ablation device for treatment before it is inserted and positioned within the patient.

At step 1006, the at least one, and preferably two, positioning elements are compressed by pushing forward the outer sheath using the slider for the outer sheath. A distal end of the catheter sheath, during placement, sits snugly up against the olive-shaped distal tip. The distal end of the catheter, with the compressed positioning elements, is then slid inside a cervix of the patient, at step 1008. At step 1010, the outer sheath of the catheter is pulled back using the corresponding slider, thereby deploying or expanding the positioning elements. The outer sheath is pulled back until the proximal positioning element abuts the internal os and some resistance is sensed by the physician. At step 1012, the cervical stabilizer is slid into its position. The cervical stabilizer is pushed by the physician until a resistance is felt. The stabilizer provides stability to the distal section of the catheter as well as tactile control to the physician.

At step 1014, at least one vapor ablation cycle is initiated. In some embodiments, the vapor is delivered for a period of time that is less than 60 seconds, and preferably for a period of time less than 30 seconds. A thermocouple positioned on an outer surface of the catheter sheath monitors the cervix temperature. The physician may choose to turn off the vapor if the thermocouple communicates that the temperature within the cervix is high. Further, the ablation device automatically turns off the vapor if the temperature is higher than a pre-defined threshold.

At step 1016, the cervical stabilizer is pulled back. At step 1018, the outer sheath of the catheter is pushed forward using the corresponding slider to compress the positioning elements. At step 1020, the catheter is finally pulled out of the patient. In some cases, the aspiration catheter is subsequently inserted to clean the ablation area.

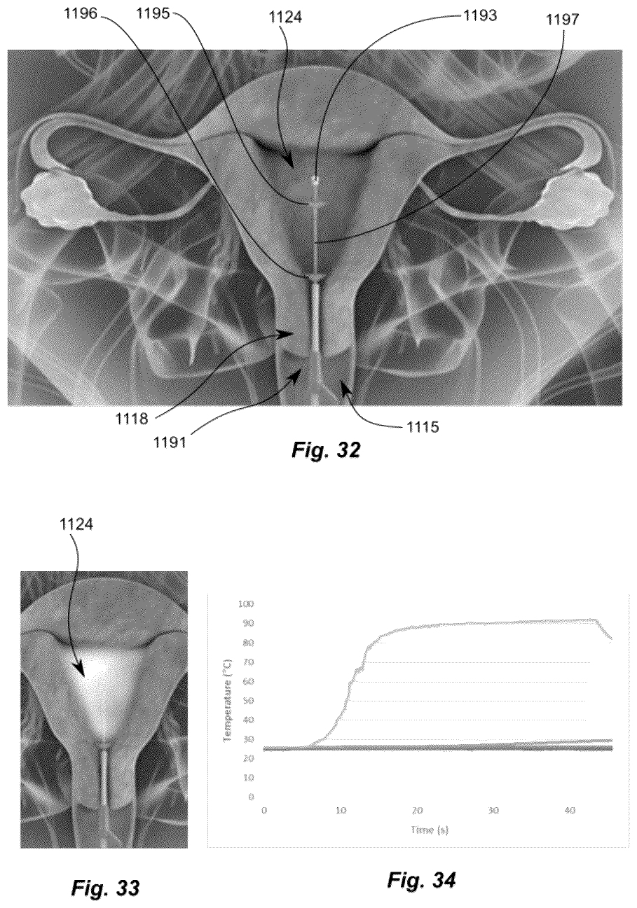

FIGS. 32 and 33 show an embodiment of catheter 100 positioned positioned within uterus 1124. Distal ends of ablation catheters 1191 having distal olive tips 1193, positioning elements 1195, and a plurality of ports 1197 is inserted through a patient's vagina canal 1115 and into and through a portion of the patient's cervix 1111. During delivery of the catheter's distal end, distal hood 1195, inner catheter shaft, and a proximal hood 1196 are all disposed within catheter shaft such that olive tip 1193 comprises the distal end of the catheter. Olive tip 1193 is configured to be soft and atraumatic to the vaginal canal 1115, external cervical os, and cervix 1118 during positioning. During deployment, inner catheter shaft is extended from catheter shaft, through the cervix 1118 and into uterus 1124, such that inner catheter shaft is positioned within uterus 1124 proximate a fibroid/tumor/lesion that is required to be treated with ablation. Proximal hood 1196 is deployed proximate an internal cervical os to firmly position the inner catheter shaft within the uterus 1124. The olive tip 1193 is rounded and bulbous and configured to be atraumatic to body tissues. Openings in olive tip 1193 provide an exit for steam out distal to the positioning element 1195 during ablation. Ports 1197 and openings in olive tip 1193 are then used to deliver steam or vapor to ablate the target area. FIG. 33 shows the presence of steam during an ablation procedure, where the steam is output by plurality of ports 1197 as well as openings in olive tip 1193. In some embodiments, a 40 second cycle of vapor ablation is delivered to the uterus.

FIG. 34 is a graph showing temperature in the uterine cavity, the cervical canal and the external fundus, as measured at at least three different points in time during an ablation procedure using the embodiments of the present specification. In the illustrated graph, a first line 1202 is the reading from a thermocouple within the uterine cavity, a second line 1204 is from the cervical canal, a third line 1206 is from the left external fundus, and a fourth line 1208 is from the right external fundus. As depicted in the illustrated graph, no thermal injury occurs in the cervical canal and the external fundus.

Using embodiments of the present specification for endometrial ablation, 100% ablation of the endometrial tissue can be obtained in all regions of the uterus without causing any thermal injury to the cervix or the fallopian tubes. Tests conducted using embodiments of the present specification with ablation time of 90 seconds achieved the minimal global triphenyl tetrazolium chloride (TTC)-negative endomyometrial ablation depth of 1.64 mm (min: max 0-3 mm) and the maximum global TTC-negative endomyometrial ablation depth of 3.50 mm (min: max 1-5 mm). TTC is a common enzymatic tetrazolium-based viability stain which detects high-temperature ablations via thermal cellular and extracellular protein denaturation. Results of the staining demonstrated complete (100%) full thickness TTC-negative endometrial ablation in all regions of the uterus in all nine samples regardless of the duration of ablation. The median upper cavity (fundus and corpus), lower uterine cavity, and right and left cornua percentages of TTC-negative surface endometrial treatment were 99.4% (range: 95-100%), 100% (range: 100-100%), 100% (range: 100-100%), and 100% (range: 100-100%) (data not shown). For the samples treated for 120 seconds, the mean minimal global TTC-negative endomyometrial ablation depth was 1.78 mm (SD 1.533, min: max 0-9.7 mm). The mean maximum global TTC negative endomyometrial ablation depth was 12.18 mm (SD 3.92; min: max 1-20 mm). For the sample treated for 45s/30s/45s (90s total ablation), the min: max ablation depth was 1.07:4.79 mm, at 60s/30s/45s (105s total ablation) the min: max ablation depth was 1.36:6.71 mm, and at 90 seconds continuous the min: max ablation depth was 1.64:3.50 mm. Therefore, embodiments achieve complete (100%) distribution of vapor within the uterine cavity.

Imaging capabilities may be added to the ablation systems used for benign prostatic hyperplasia (BPH), abnormal uterine bleeding (AUB), over-active bladder (OAB), and for any other tissue ablation processes described in the embodiments of the present specification. In embodiments, the imaging capabilities are provided in the form of an integrated optical chip with the ablation system or as a coaxial fiber optical wire with the sheath of the catheter of the ablation system.

The above examples are merely illustrative of the many applications of the system of the present invention. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed:

1. An ablation catheter for ablating endometrial tissue of a patient, comprising:
an outer sheath having a first diameter and a length extending between a proximal end and a distal end, the outer sheath configured with at least one opening;
an inner shaft having at least one lumen, wherein the lumen is configured to receive a volume of fluid;
at least one positioning element positioned proximate a proximal or distal end of the inner shaft, wherein the at least one positioning element is configured to expand from a first compressed delivery configuration to a second expanded deployed configuration;
at least one first port positioned on the inner shaft;
an atraumatic tip positioned on a distal end of the inner shaft, wherein the atraumatic tip comprises at least one second port in fluid communication with the inner shaft;
at least one heating component positioned within the lumen, wherein the at least one heating component comprises at least one electrode positioned circumferentially around a central core, and wherein the at least one heating component is configured to convert the volume of fluid received by the lumen to a vapor that exits from the at least one first port and the at least one second port;
and
a handle coupled to a proximal end of the inner shaft.

2. The ablation catheter of claim 1, wherein the at least one electrode comprises at least one array of electrodes.

3. The ablation catheter of claim 2, wherein the array of electrodes comprises at least two electrodes having tapered proximal ends, wherein the at least two electrodes are configured to be positioned circumferentially around the central core such that the two tapered proximal ends are offset from one another.

4. The ablation catheter of claim 1, wherein a wall of the inner shaft comprises at least one insulation layer.

5. The ablation catheter of claim 1, wherein the core is tapered at a proximal end of the core.

6. The ablation catheter of claim 1, wherein the at least one positioning element comprises a wire mesh structure.

7. The ablation catheter of claim 1, wherein the handle comprises at least one sliding button to control movement of the sheath to control deployment of the at least one positioning element.

8. The ablation catheter of claim 1, further comprising a stabilizer positioned coaxially around an outer circumference of the sheath on a proximal side of the first positioning element, wherein the stabilizer is configured to stabilize and maneuver the sheath during insertion inside the uterine cavity, the stabilizer comprising:
a first proximal section;
a second section attached to and perpendicular the first section;
a third section attached to a distal end of, and at an angled orientation relative to, the second section, wherein the third section extends in a distal direction relative to the second section; and
a fourth section extending in a distal direction relative to the second section, wherein the fourth section is attached to a side of a distal end of the third section, and wherein the fourth section is oriented parallel to the second section, and wherein the fourth section is configured to be hollow to accommodate passage of the sheath.

9. An ablation system for ablating endometrial tissue of a patient, comprising:
a catheter comprising:
an outer sheath having a first diameter and a length extending between a proximal end and a distal end, the outer sheath configured with at least one opening;
an inner shaft having at least one lumen, wherein the lumen is configured to receive a volume of fluid;
at least one positioning element positioned proximate a proximal or distal end of the inner shaft, wherein the at least one positioning element is configured to expand from a first compressed delivery configuration to a second expanded deployed configuration;
at least one first port positioned on the inner shaft;
an atraumatic tip positioned on a distal end of the inner shaft, wherein the atraumatic tip comprises at least one second port in fluid communication with the inner shaft;
at least one heating component positioned within the lumen, wherein the at least one heating component comprises at least one electrode positioned circumferentially around a central core, and wherein the at least one heating component is configured to convert the volume of fluid received by the lumen to a vapor that exits from the at least one first port and the at least one second port; and
a handle coupled to a proximal end of the inner catheter shaft;
a fluid reservoir configured to contain the volume of fluid and in fluid communication with the at least one lumen;
a pump configured to pump fluid from the fluid reservoir into the at least one lumen; and
a controller having at least one processor, wherein the controller is configured to deliver an electrical current to the at least one electrode and to cause the volume of fluid to pass into the lumen from the fluid reservoir when activated.

10. The ablation system of claim 9, further comprising a power source positioned in the controller.

11. The ablation system of claim 9, wherein the pump comprises a syringe pump.

12. The ablation system of claim 9, wherein the at least one electrode comprises at least one array of electrodes.

13. The ablation system of claim 9, wherein the core is tapered at a proximal end of the core.

14. The ablation system of claim 9, wherein the controller comprises a foot pedal or switch configured to allow a user to control a flow of fluid to the lumen.

15. The ablation system of claim 9, wherein the at least one positioning element comprises a wire mesh structure.

16. The ablation system of claim 9, wherein the handle comprises at least one sliding button to control movement of the sheath to control deployment of the at least one positioning element.

17. The ablation system of claim 9, further comprising a stabilizer positioned coaxially around an outer circumference of the sheath on a proximal side of the first positioning element, wherein the stabilizer is configured to stabilize and maneuver the sheath during insertion inside the uterine cavity, the stabilizer comprising:
a first proximal section;
a second section attached to and perpendicular the first section;
a third section attached to a distal end of, and at an angled orientation relative to, the second section, wherein the third section extends in a distal direction relative to the second section; and
a fourth section extending in a distal direction relative to the second section, wherein the fourth section is attached to a side of a distal end of the third section, and wherein the fourth section is oriented parallel to the second section, and wherein the fourth section is configured to be hollow to accommodate passage of the sheath.

18. A kit comprising multiple components of an ablation system, the kit comprising:
an ablation catheter comprising:
an outer sheath having a first diameter and a length extending between a proximal end and a distal end, the outer sheath configured with at least one opening;
an inner shaft having at least one lumen, wherein the lumen is configured to receive a volume of fluid;
at least one positioning element positioned proximate a proximal or distal end of the inner shaft, wherein the at least one positioning element is configured to expand from a first compressed delivery configuration to a second expanded deployed configuration;
at least one first port positioned on the inner shaft;
an atraumatic tip positioned on a distal end of the inner shaft, wherein the atraumatic tip comprises at least one second port in fluid communication with the inner shaft;
at least one heating component positioned within the lumen, wherein the at least one heating component comprises at least one electrode positioned circumferentially around a central core, and wherein the at least one heating component is configured to convert the volume of fluid received by the lumen to a vapor that exits from the at least one first port and the at least one second port; and
a handle coupled to a proximal end of the inner catheter shaft;
at least one pump configured to pump fluid from a fluid reservoir into the at least one lumen;
an aspiration catheter;
a substrate comprising a plurality of spaces configured to receive the ablation catheter, at least one pump, and aspiration catheter, wherein at least one first cover portion of the substrate is configured to cover and protect at least one component of the ablation catheter;
a base configured to receive the substrate; and
a lid configured to be positioned over the substrate and secured to the base.

19. The kit of claim 18, wherein the at least one first cover portion is partially cut out from a remainder of the substrate and is formed by folding the at least one first cover portion over the at least one component of the ablation catheter.

20. The kit of claim 18, further comprising at least one second cover portion and at least one third cover portion, wherein the at least one first, second, and third cover portions are configured to engage with each other to form an enclosure to cover the at least one component of the ablation catheter.

21. The kit of claim 18, wherein the lid comprises a plurality of recessed cavities that are recessed according to shapes of the ablation catheter, at least one pump, and aspiration catheter.

22. The kit of claim 18, wherein at least one space of the plurality of spaces of the substrate is configured to receive the ablation catheter comprising the at least one positioning element configured to expand from a first compressed delivery configuration to a second expanded deployed configuration, wherein the at least one positioning element is in the second expanded deployed configuration when the ablation catheter is positioned within the at least one space, and wherein the at least one first cover portion is configured to cover and protect the at least one positioning element in the second expanded deployed configuration.
